(19) United States
(12) Patent Application Publication
(10) Pub. No.: US 2025/0270594 A1
(43) Pub. Date: Aug. 28, 2025

(54) RARE EARTH ELEMENT PHOSPHATES AND PROCESS FOR MAKING

(71) Applicant: BATTELLE MEMORIAL INSTITUTE, Columbus, OH (US)

(72) Inventors: Katarzyna Kucharzyk, Columbus, OH (US); Ray Henson, Columbus, OH (US); Scott Banta, New York, NY (US)

(21) Appl. No.: 19/062,405

(22) Filed: Feb. 25, 2025

Related U.S. Application Data
(60) Provisional application No. 63/557,750, filed on Feb. 26, 2024.

(51) Int. Cl. C12P 3/00; C12N 9/16

(52) U.S. Cl. CPC C12P 3/00; C12N 9/16; C12Y 301/03002

(57) ABSTRACT

The disclosure relates to rare earth element (REE) phosphates and methods for making the same. The REE phosphates can be formed by combining a REE sulfate with a phosphate source and a biological catalyst, for example an acid phosphatase.

### RELATED APPLICATIONS

This application claims the benefit of U.S. Provisional Application No. 63/557,750, filed Feb. 26, 2024, the entire disclosure of which is incorporated herein by reference.

### GOVERNMENT SUPPORT

This invention was made with government support under Grant Number FA8650-22-C-7213 awarded by the Department of Defense, specifically Defense Advanced Research Projects Agency (DARPA). The government has certain rights in the invention.

### INCORPORATION BY REFERENCE OF MATERIAL SUBMITTED ELECTRONICALLY

Incorporated by reference in its entirety is a computer-readable amino acid sequence listing submitted concurrently herewith and identified as follows: 3000 bytes xml file named “920006404428.xml” created on Feb. 17, 2025.

### BACKGROUND

The phosphate forms of Rare Earth Elements (REEs) are sought after by the industrial communities due to their use as Environmental Barrier Coatings, or EBCs. EBCs are used to seal parts in the engines and structural components of rockets, hypersonic jets, and other space-bound vehicles. The coatings protect parts from harsh operating conditions such as high temperatures, supersonic speeds, intense stress, severe oxidation and corrosion. REE silicates are the current choice for EBCs used to coat the silicon carbide-based ceramic matrix materials in state-of-the-art jet engines, but these materials are problematic, and subject to performance degradation. As an alternative, EBCs made from multicomponent REE phosphates instead of silicates offer possibilities in designing future EBCs and extending their performance.

Interest in production of REE phosphates is increasing due to their potential applications for optical materials including laser, phosphors and anti-UV materials for UV shielding that mostly apply REE-phosphate amorphous forms. Cerium (Ce) and Terbium (Tb) can form tri (+3) and tetravalent (+4) ions with the most stable phases formed at room temperature which aids crystallization into the phosphates. However, the room temperature crystallization must be followed by high temperature (473-1373K for 5 hours) calcination reaction in order to achieve stable crystals. Often, a REE-mono (REE-PO₄) and orthophosphate (REE-PO₄³⁻) mixture is formed.

The current state of the art for formation of phosphorylated forms of REEs includes chemical reaction steps performed in presence of strong acid e.g., sulfuric acid, with phosphate group donor, such as sodium pyrophosphate. To achieve monophosphate or/and orthophosphate—REE crystals final reaction steps are performed at high temperatures that involve additional energy consumption driving an increase of total production cost.

### SUMMARY

In one aspect, the disclosure provides a process for forming a rare earth element (REE) phosphate, the process comprising:
(a) contacting an acid phosphatase with a phosphate source in a solution, thereby generating free phosphate;
(b) contacting an REE sulfate or an REE chloride with the free phosphate formed in step (a), thereby forming an REE phosphate; and
(c) isolating the REE phosphate formed in step (b).

In certain aspects, the formed REE phosphate is used in a coating.

### BRIEF DESCRIPTION OF THE DRAWINGS

FIG. 1A The effect of enzyme addition and controls on Europium (Eu) time-resolved fluorescence (proxy for Eu in solution) with EDTA.

FIG. 1B The effect of enzyme addition and controls on Europium (Eu) time-resolved fluorescence (proxy for Eu in solution) without EDTA.

FIG. 2 The effect of acid phosphatase on Eu time-resolved fluorescence (proxy for concentration of Eu in solution).

FIG. 3 The yield of TbPO₄ from Tb₂(SO₄)₃ using enzymatically driven precipitation. The yield was calculated using ICP-MS measurements of Tb³⁺ concentration in supernatant and redissolved precipitate in nitric acid. Bars represent the average of three replicates and error bars are one standard deviation.

FIGS. 4A-C Scanning Electron Microscopy/Energy Dispersive X-ray Spectroscopy (SEM-EDS) images of Tb-phosphate product generated in cell-free reactions with glycerol phosphate.

FIGS. 5A and B Fluorescence emission spectrum (from 450 to 650 nm) from excitation at 350 nm of the biologically prepared material (FIG. 5A) as compared to a published reference material (FIG. 5B) from Tran et al.

FIG. 6 TGA of synthesized material.

FIG. 7A X-ray diffraction (XRD) pattern of enzyme prepared material.

FIG. 7B XRD identified the orthophosphate form (˜40%) and the triphosphate form (˜60%).

### DETAILED DESCRIPTION

This disclosure described a controlled room temperature crystallization of REEs by application of biological catalyst without extensive acid waste streams, extensive heating, and controllable crystallization. As demonstrated by the data, the reaction is performed in biologically relevant pH conditions, and the change in the temperature conditions facilitates change in the reaction kinetics of the enzyme and speed of REE-phosphate crystal formation, which can be beneficial for formation of different crystal structures. It is also possible that different REEs may produce different crystal structures of their corresponding phosphate forms. Thus, by changing a temperature conditions of a reaction and activity of enzyme towards different substrates, the difference in activity and rate of precipitation between two rare earth elements, lanthanum (La) and Tb, is demonstrated, indicating that this process can be used to isolate REEs as a phosphate salt.

In certain aspects, REE sulfates are converted into REE-phosphates at room temperature to form a crystallized REE containing material. This room temperature reaction is facilitated by application of acid phosphatase enzyme (EC 3.1.3.2, systematic name phosphate-monoester phosphohydrolase (acid optimum)) to hydrolase a phosphate group from a donor compound (e.g., ATP or glycerol phosphate) for final production of crystallized REE phosphate material. While this disclosure demonstrates this approach in a cell free manner, the phosphatase could also perform this chemistry intracellularly to form REE-phosphate nanoparticles. Specific microorganisms that can be applied for intracellular conversion of REE-ions to their phosphate forms are, for example: Methylorubum extorquens, Acidithiobacillus ferooxidans, Acidithiobacillus thiooxidans, Sulfolobus acidocaldarius, Shewanella oneidensis, Pseudomonas putida, Corynebacterium glutamicum, Rhodococcus jostii, Eschericia coli, Saccharomyces cerevisiae, Yarrowia lipolytica, among many others.

This enzymatic phosphorylation process can be performed with many phosphate sources, including pyrophosphate, adenosine triphosphate (ATP), and glycerophosphate. Other phosphate group donors can be applied in this process based on the pH of the reaction environment, selectivity, and the DNA sequence of the acid phosphatase enzyme.

This concept was demonstrated by using pyrophosphate, ATP, and glycerophosphate as phosphate group donors and a subsequent conversion of REEs, specifically europium (Eu), lanthanum (La), and terbium (Tb) sulfates into their phosphate forms using a commercial off the shelf acid phosphatase. The production of terbium phosphate crystals and its corresponding characterization with several advanced analytical methods including fluorescence spectroscopy validating formation of Tb-phosphate crystalline material is also demonstrated.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or,

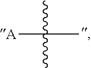

where each of “-*”, “-**”, and

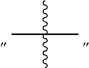

represents a bond to A and the point of covalent bond attachment to B. Alternatively, in some embodiments, the portion of A-B defined by the group or chemical structure B can be represented by “*-B”, “**-B”, or

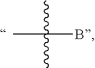

where each of “-*”, “-**”, and

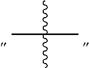

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub- combination of chemical groups was individually and explicitly disclosed herein.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy,-OCO-CH2-O- alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted. As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “cycloalkyl” refers to a saturated or partially saturated, monocyclic or polycyclic mono-valent carbocycle. The term “cycloalkylene” refers to a saturated or partially saturated, monocyclic or polycyclic di-valent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include mono-valent radicals of the following entities, while cycloalkylene groups include di-valent radicals of the following entities, in the form of properly bonded moieties:

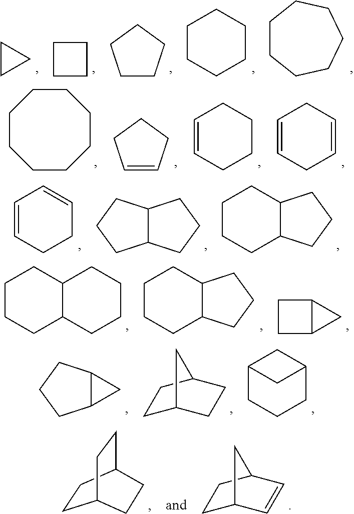

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a di-valent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3-to 12-membered), or 3 to 7 ring atoms (3-to 7-membered), or 3 to 6 ring atoms (3-to 6-membered), or 4 to 6 ring atoms (4-to 6-membered), or 5 to 7 ring atoms (5-to 7-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group on a carbon ring member or up to two oxo groups on sulfur ring members. Illustrative examples of heterocycloalkyl groups include mono-valent radicals of the following entities, while heterocycloalkylene groups include di-valent radicals of the following entities, in the form of properly bonded moieties:

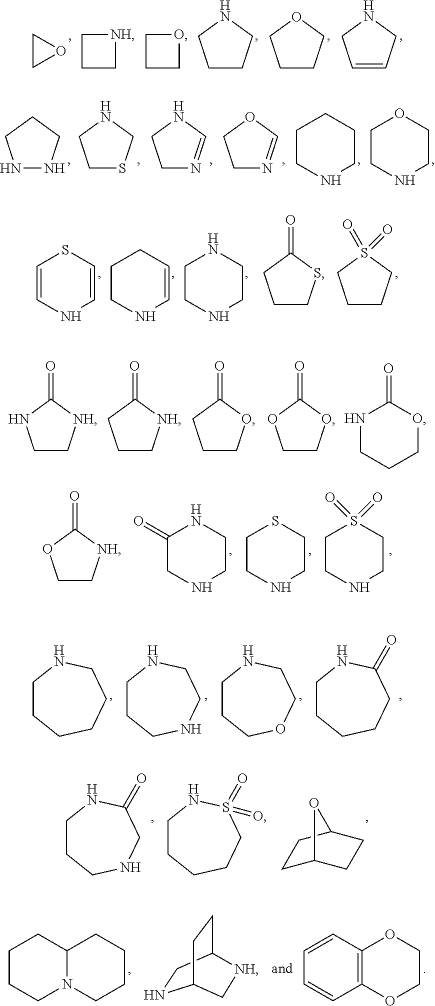

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆alkyl group, for example, contains from one to six carbon atoms in the chain.

The term “sulfate” is art-recognized and refers to the group (SO₄)²⁻, or an acceptable salt thereof.

The term “phosphate” is art-recognized and refers to the group (PO₄)³⁻, or an acceptable salt thereof.

The term “sulfonamide” is art-recognized and refers to the group represented by the general formulae

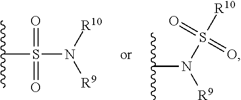

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

“Acceptable salt” or “salt” is used herein to refer to an acid addition salt or a basic addition salt which is suitable for or compatible with the compounds or a desired treatment.

The term “acceptable acid addition salt” as used herein means any non-toxic organic or inorganic salt of any base compounds described herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I or II are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I or II for laboratory use, or for subsequent conversion to a pharmaceutically acceptable acid addition salt.

The term “acceptable basic addition salt” as used herein means any non-toxic organic or inorganic base addition salt of any acid compounds described herein or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

In one aspect of the disclosure, a process is used to convert particular salts of rare earth elements (REEs) to a desired different REE salt. For example, the processed described herein can be used to convert a REE sulfate, chloride, or a mixture thereof to an REE phosphate. The REE phosphate may precipitate out of solution into a crystalline REE phosphate that can be used for a variety of applications, such as, for example, a composition of for a coating. In certain aspects, the initial REE salts (e.g., sulfate, chloride, or combination thereof) can be in complex mixtures such as waste or mining solutions such that the described process functions to isolate REEs as a phosphate salt. In some embodiments, the REE phosphates are formed in the present of a phosphate source and an enzyme, such as a phosphatase. In some embodiments, the process comprises the steps of:
(a) contacting an phosphatase (e.g., an acid phosphatase) with a phosphate source in a solution, thereby generating free phosphate;
(b) contacting an REE sulfate or an REE chloride with the free phosphate formed in step (a), thereby forming an REE phosphate;
(c) isolating the REE phosphate formed in step (b).

In some embodiments, the REE is Scandium (Sc), Yttrium (Y), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), or Lutetium (Lu), and is preferably Europium (Eu), Lanthanum (La), Terbium (Tb), or a mixture thereof. The REE sulfate, chloride, or a combination thereof may be in complex mixture in the solution. Prior to interacting with the free phosphate, the REE sulfate, chloride, or combination thereof is substantially or entirely dissolved in the solution such that in certain embodiments the REE is fully dissociated from the corresponding starting anion (e.g., chloride or sulfate).

In some embodiments, the solution (i.e., the solution present in steps (a)-(c)) is at a pH of about 1 to about 7, and is preferably about 4 to about 5. The solution may comprise a buffer salt(s). In some embodiments, the solution comprises sodium acetate, citrate, malonate, glycine, Tris, MES (2-(N-morpholino)ethanesulfonic acid), Homopiperazine-1,4-bis(2-ethanesulfonic acid, (Homo-PIPES), sodium chloride, potassium chloride, sulfuric acid, hydrochloric acid, glutamic acid, histidine, malate, or a mixture thereof Certain buffers, for example, acetate, can affect the activity of the phosphatase (e.g., acid phosphatase), chelate the REE, or both. In some embodiments, the solution includes Ethylenediaminetetraacetic acid (EDTA), Ethyleneglycol-bis(β-aminoethyl)-N,N,N′,N′-tetraacetic acid (EGTA), or a mixture thereof. In certain embodiments, the solution does not include exogenous zinc, iron, or mangansese. In certain preferred embodiments, the solution includes a chelator (e.g., EDTA or EGTA) and does not include exogenous zinc, iron, or mangansese or is substantially free from zinc, iron, or mangansese. Without being bound by theory, the REE may be cooperating with the phosphatase to generate the free phosphate that subsequently interacts with the REE.

The phosphate source interacts with the phosphatase to yield a free phosphate that can interact with the REE salts (e.g., a sulfate, chloride, or a mixture thereof). For example, the phosphate source can include a cleavable phosphate that is cleaved by the phosphatase to yield a free phosphate. Certain phosphate sources, such as ATP, contain three phosphates such that they may yield one, two, or three free phosphates, or alternatively yield a pyrophosphate and a free phosphate. When phosphate sources contain multiple possible phosphates for reactions, undesired side products can occur with the phosphate donor source. For example, a reaction can occur with both adenosine diphosphate as well as adenosine triphosphate under certain conditions. Certain preferred phosphate sources, such as glycerophosphate, contain only a single phosphate per molecule and can therefore only yield a single free phosphate. In some embodiments, the phosphate source is pyrophosphate, adenosine triphosphate, a cycloalkyl phosphate, a heterocycloalkyl phosphate, an alkyl phosphate (e.g., a hydroxy substituted C₁-C₆ alkyl such as glycerophosphate), each of which is optionally substituted with, for example, a hydroxy, and is preferably a hydroxy-substituted alkyl phosphate such as glycerophosphate.

In some embodiments, the phosphate source (e.g., glycerophosphate) is present in the solution at the beginning of the process (i.e., before action by the phosphatase) at a concentration of about 1 μM to about 1 M, preferably at about 0.5 mM to about 5 mM. It should be understood that this starting concentration of phosphate source will decrease as the phosphatase cleaves the phosphate from the source during the course of the process. In some embodiments, the concentration of the phosphate source is such that the total yielded free phosphate is comparable to the amount of REE salt (e.g., chloride or sulfate) at the beginning of the process.

In certain embodiments, the phosphatase is an acid phosphatase. Examples of acid phosphatases include purple acid phosphatase (NP_001275256.1), MP Biomedicals Acid Phosphatase, Wheat Germ (cat. #ICN15387690) available from Fisher Scientific; phosphatase, acid from potato (cat. #P3752) available from Millipore Sigma; phosphatase, acid from sweet potato (cat. #P1435) available from Millipore Sigma; phosphatase, acid from potato (cat. #5245291KU) available from Millipore Sigma; and prostatic acid phosphatase (cat. #AG60) available from Millipore Sigma. In certain embodiments, the phosphatase has at least about 90%, at least about 95%, or at least about 99% sequence identity to purple acid phosphatase (NP_001275256.1), MP Biomedicals Acid Phosphatase, Wheat Germ (cat. #ICN15387690) available from Fisher Scientific; phosphatase, acid from potato (cat. #P3752) available from Millipore Sigma; phosphatase, acid from sweet potato (cat. #P1435) available from Millipore Sigma; and prostatic acid phosphatase (cat. #AG60) available from Millipore Sigma. In certain embodiments, the phosphatase includes a mutation relative to purple acid phosphatase (NP_001275256.1), MP Biomedicals Acid Phosphatase, Wheat Germ (cat. #ICN15387690) available from Fisher Scientific; phosphatase, acid from potato (cat. #P3752) available from Millipore Sigma; phosphatase, acid from sweet potato (cat. #P1435) available from Millipore Sigma; or prostatic acid phosphatase (cat. #AG60) available from Millipore Sigma, and the mutation is present in a conserved domain such as an active site that is configured to bind to two metal ions (e.g., manganese, iron, or zinc) that are coordinated with octahedral geometry by a cage of histidine, aspartate, and asparagine residues. In certain embodiments, the REE may interact with (e.g., bind) the phosphatase prior to being contacted by the free phosphate.

In certain embodiments, the phosphatase is a purple acid phosphatase according to NCBI Reference: NP_001275256.1 (SEQ ID NO.: 1). Certain derivatives or mutants of the phosphatase (e.g., purpose acid phosphatase) may also be suitable, for example those having at least about 90%, at least about 95%, or at least about 99% sequence similarity to NP_001275256.1 (SEQ ID NO.: 1). In certain embodiments, the phosphatase has one, two, three, or four mutations relative to the commercially available phosphatase (e.g., purple acid phosphatase NP_001275256.1 (SEQ ID NO.: 1)). The mutations may preferably in a conserved domain having active site that includes two metal ions (usually manganese, iron, or zinc) coordinated with octahedral geometry by a cage of histidine, aspartate, and asparagine residues.

The phosphatase (e.g., an acid phosphatase) may have desired activity at pH less than 7, for example at a pH of about 1 to about 7 or about 1 to about 5.5. In certain preferred embodiments, the phosphatase is an acid phosphatase and has desirable activity in NaOAc at a pH of about 5. In certain embodiments, the phosphatase (e.g., acid phosphatase) is present at about 0.001 g/L to about 0.5 g/L and is preferably about 0.01 g/L to about 0.1 g/L.

In some aspects, the rate of cleavage (i.e., the yielding of the free phosphate) is such that the subsequent steps of contacting the REE chloride or sulfate occur at a preferred rate. For example, in some aspects the production of the free phosphate in step (a) is the rate limiting step of the process and allows for preferred rates of precipitation of the REE phosphate. In some embodiments, the REE phosphate is formed at a rate of about 0.001 g/l/hr to about 100 g/L/hr, preferably about 0.1-g/L/hr to about 1 g/L/hr. Certain rates of formation of the REE phosphate and precipitation may be more likely to yield crystalline REE phosphate.

In some embodiments, the process is performed in a cell-free solution (i.e., in a solution in the absence of cells such as bacterial cells that produce and excrete phosphatase). For example, the process may be performed by adding recombinant phosphatase (e.g., acid phosphatase) to a solution. Alternatively, the process can be performed in the presence of cells (i.e., cells, such as bacterial cells, that are added to the solution), where the cells can excrete a phosphatase (e.g., acid phosphatase) that then interact with the phosphate source. Or the process can be performed in the presence of cells (e.g., cells, such as bacterial cells, that are added to the solution), where the REEs are present inside the cells (e.g., cells, such as bacterial cells), and an intra-cellular phosphatase interacts with the phosphate source to yield the free phosphate inside the cell. This intra-cellular process may yield nanoparticles of REE phosphates.

In some embodiments, the process is performed at a temperature of about 4° C. to about 50° C. The temperature may be selected to provide preferred rates of formation of the free phosphate, precipitation of the REE phosphate, or both. In some embodiments, the process is performed at a temperature of about 20° C. to about 40° C., and is preferably at about 37° C. In certain embodiments, the process is performed at a temperature less than about 100° C.

In some embodiments, the process include a step of isolating the yielded REE phosphate. During the course of the process, the produced REE phosphate may precipitate out of solution, optionally in crystalline form. In certain embodiments, the process includes the step of filtering the solid (e.g., crystalline) REE phosphate. In certain embodiments, the produced REE phosphate is calcined to generate the anhydrous form.

In some embodiments, the formed REE phosphate is a crystalline material. In some embodiments, the REE phosphate (e.g., the crystalline REE phosphate) is used in a coating.

### EMBODIMENTS

1. A process for forming a rare earth element (REE) phosphate, the process comprising:
   (a) contacting an acid phosphatase with a phosphate source in a solution, thereby generating free phosphate;
   (b) contacting an REE sulfate or an REE chloride with the free phosphate formed in step (a), thereby forming an REE phosphate; and
   (c) isolating the REE phosphate formed in step (b).
2. The process of embodiment 1, wherein the REE is Scandium (Sc), Yttrium (Y), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), or mixtures thereof.
3. The process of embodiment 1 or 2, wherein the REE is Europium (Eu), Lanthanum (La), or Terbium (Tb).
4. The process of any one of the preceding embodiments, wherein the pH of the solution in step (a) is about 1 to about 7.
5. The process of any one of the preceding embodiments, wherein the pH of the solution in step (a) is about 4 to about 5.
6. The process of any one of the preceding embodiments, wherein the solution is a buffered solution.
7. The process of any one of the preceding embodiments, wherein the solution comprises a sodium acetate, citrate, malonate, glycine, Tris, MES (2-(N-morpholino) ethanesulfonic acid), Homopiperazine-1,4-bis(2-ethanesulfonic acid, (Homo-PIPES), sodium chloride, potassium chloride, sulfuric acid, hydrochloric acid, glutamic acid, histidine, or malate.
8. The process of any one of the preceding embodiments, wherein the solution comprises a EDTA or EGTA.
9. The process of any one of the preceding embodiments, wherein the phosphate source is pyrophosphate, adenosine triphosphate, or a hydroxy-substituted C₁-C₆ alkyl such as glycerophosphate.
10. The process of any one of the preceding embodiments, wherein the phosphate source (e.g., glycerophosphate) provides a single phosphate per molecule.
11. The process of any one of the preceding embodiments, wherein the phosphate source is present at a concentration of about 1 μM to about 1 M when the process begins.
12. The process of any one of the preceding embodiments, wherein the process is performed in a cell-free solution.
13. The process of any one of the preceding embodiments, wherein the acid phosphatase is present at a concentration of 0.02 g/L or lower.
14. The process of any one of the preceding embodiments, wherein the process is performed in the presence of a cell.
15. The process of embodiment 14, wherein the cell generates the acid phosphatase.
16. The process of any one of the preceding embodiments, wherein the REE phosphate is formed in a bacterial cell.
17. The process of embodiment 16, wherein the cell generates the acid phosphatase.
18. The process of any one of the preceding embodiments, wherein the process is performed at a temperature of about 4° C. to about 50° C.
19. The process of any one of the preceding embodiments, wherein the process is performed at a temperature of about 20° C. to about 40° C.
20. A process for converting Rare Earth Elements (REEs) to phosphate forms, the method comprising:
   adding a phosphatase to a sample comprising an REE salt, and
   recovering an REE phosphate from the sample.
21. The process of any one of the preceding embodiments, wherein the phosphatase comprises a conserved domain with an active site consisting of two metal ions (e.g., manganese, iron, or zinc) coordinated with octahedral geometry by a cage of histidine, aspartate, and asparagine residues.
22. The process of any one of the preceding embodiments, wherein the phosphatase comprises the reference sequence NP_001275256.1 (SEQ ID NO.: 1).
23. The process of any one of embodiments 1-21, wherein the phosphatase comprises a phosphatase with at least about 95% or at least about 99% sequence similarity to the reference sequence NP_001275256.1 (SEQ ID NO.: 1).
24. The process of any one of the preceding embodiments, wherein the REE phosphate is formed at a rate of about 0.001 g/l/hr to about 100 g/L/hr, preferably about 0.1-g/L/hr to about 1 g/L/hr.
25. The process of any one of the preceding embodiments, wherein the step of isolating the REE phosphate yields crystalline REE phosphate.
26. A coating comprising REE phosphate produced by a process according to any one of the preceding embodiments.

### EXAMPLES

### Example 1

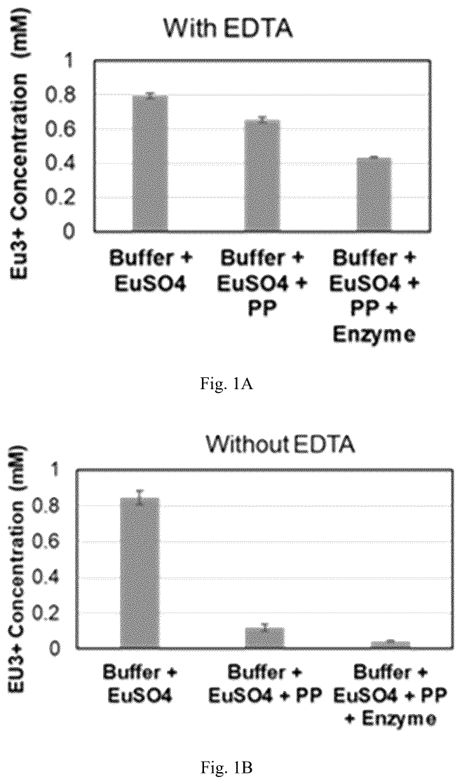

Significant reduction in fluorescence was observed for europium (Eu) after a 2-hour incubation with acid phosphatase enzyme (phosphatase, acid from potato (cat. #5245291KU) available from Millipore Sigma) (63% reduction with enzyme addition without ethylenediaminetetraacetic acid (EDTA) in presence of pyrophosphate as phosphate source) (FIGS. 1A and 1B) indicating precipitation and a reduction in aqueous concentration of europium. Potential side effects of pyrophosphate addition and EDTA during enzyme incubation further investigation with ICP-MS and other methods.

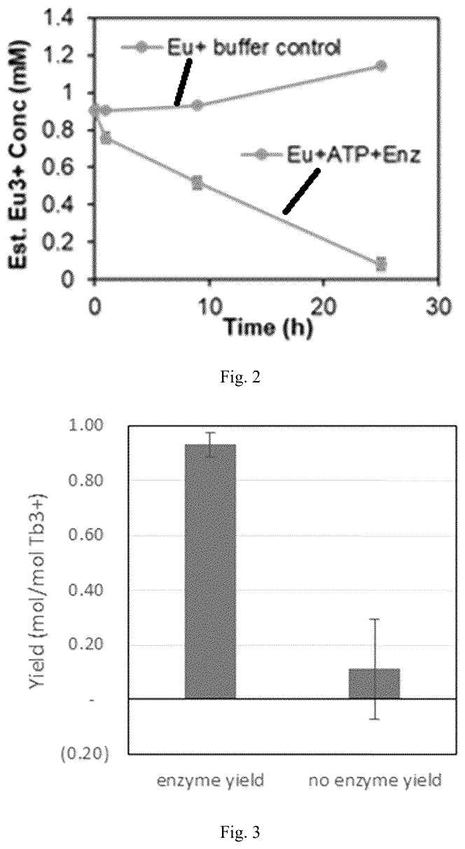

Additional tests showed significant reduction in Eu fluorescence compared to control with acid phosphatase using adenosine triphosphate as a phosphate source (FIG. 2). To validate these results, samples were submitted for ICP-OES analysis.

### Example 2

To explore the transformation of REEs by phosphorylation, an experiment was designed to perform this reaction cell-free. Because of the compatibility with downstream processing, both acid and alkaline phosphatases were tested in different buffers. Two commercial-off-the-shelf phosphatase enzymes (phosphatase, acid from potato (cat. #5245291KU) available from Millipore Sigma, and alkaline phosphatase (cat. #PI31391) available from Thermo Scientific) that operate at low pH and high pH conditions were examined. Phosphatase enzymes with different phosphate sources were then evaluated. Initial results indicated that pyrophosphate could act as a phosphate donor, but abiotic precipitation with the phosphate donor was observed in this case. These effects prompted consideration of phosphate donors that were easily cleaved by phosphatase enzymes.

ATP was then tested and some promising results were observed. However, X-Ray Diffraction (XRD) results indicated that ADP was also reacting and precipitating with the REE. Based on this information, a phosphate donor that has a single phosphate group to donate was considered. This led to the selection of glycerophosphate as a phosphate donor, of which we had the most success and observed crystalline REE phosphate formation.

For each of these experiments, Tb was the selected REE. But additional experiments validating that this approach can be used for precipitation of other REEs such as La and Eu.

The workflow focused on conversion of sulfate to phosphate form completed using cell-free enzymatic approach with a proposed equation achieving the conversion:

Tb₂(SO₄)₃+C₃H₉O₆P→(enz) TbPO₄ (prec.)+C₃H₈O₃+(SO₄)²⁻

Solution preparation was performed in 1.5 ml microcentrifuge tubes up to scales of 1 L volumes. Briefly, to prepare precipitate, 1 mM of Tb₂(SO₄)₃ was added to 200 mM sodium acetate buffer at pH=5.0 along with 1 mM glycerophosphate and 0.5 g/L enzyme and heated at 37° C. for up to 24 hours. Precipitation was not observed until heating at 37° C. No-enzyme controls were used throughout the process to confirm that no abiotic precipitation was observed.

After precipitate formation, the solution was centrifuged at 3000×g for 5 minutes, then washed thrice with distilled water to remove any remaining products such as glycerol and sulfate ions. The material was dried overnight in a chemical hood and characterized by ICP-MS, thermogravimetric analysis (TGA), mass balance to obtain yields, and other characterizations. A portion of the material was also calcined and evaluated by XRD and scanning electron microscopy (SEM-EDS) to determine morphology and atomic composition. A positive control material using sodium phosphate to precipitate Tb at pH 2 followed by calcination at 750° C. to induce crystal formation for chemical identification was prepared.

Multiple steps through the process required use of analytical methods to provide burden of proof of chemical form conversion of selected element and characterization of the generated product. Table 1 provides more detailed information on the type of analytical methods and results generated in this task.

TABLE 1
Methods for analysis of precipitate to confirm composition
and mass of generated phosphate REE.

| Method | Properties | Results |
|---|---|---|
| ICP-MS | Concentration, | Confirmed reduction of Tb in solution and |
| | composition | collection in precipitate |
| Mass | Mass yield of | 93% yield (mol TbPO4/mol Tb2(SO4)3 |
| balance | precipitate | |
| SEM-EDS | Morphology, | Confirmed phosphorus, oxygen, and |
| | elemental | terbium in precipitate |
| | composition | |
| XRD | Crystal structure, | Terbium orthophosphate and terbium |
| | chemical formula | triphosphate composition of precipitate |
| TGA | Hydrate form of | Hydrate form produced, triphosphate form |
| | precipitate | most likely produced |
| Microplate | Fluorescence | Fluorescence emission spectrum matches |
| reader | excitation | TbPO4 nanorods |
| | and emission | |

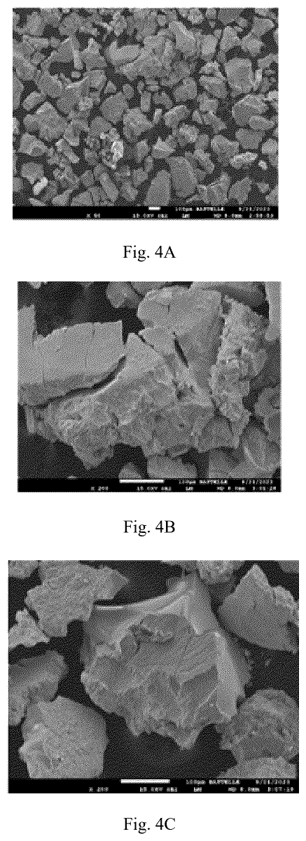

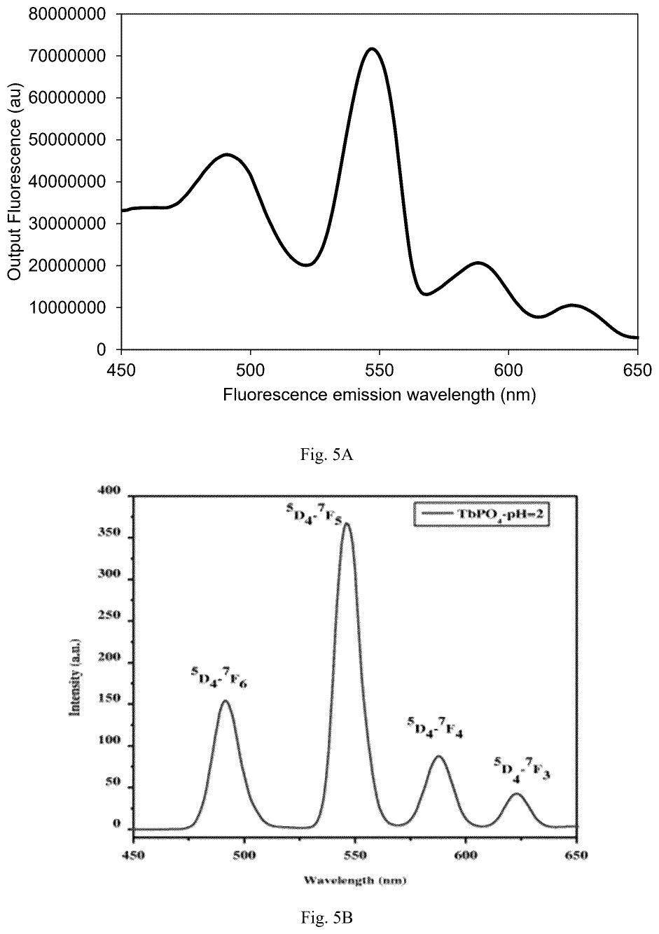

ICP-MS analysis of the solution from initial enzyme addition (t=0 hours) and after 24 hours of reaction time along with evaluation of precipitate indicated we reached a 93%+−5 yield (FIG. 3). Other measurements including mass balance using the precipitate indicate that the error in the ICP-MS measurement was ˜+−10%, and small amounts of Tb measured after washing the no enzyme control suggests some carryover, however, a trend to high yields greater than 70% at this stage is observed.

Once the experiment was executed with the abovementioned workflow, after calcining, SEM-EDS indicated bulk material contained Tb and phosphorus with trace impurities such as Na, Si, and S, suggesting minimal biomass in final material (FIGS. 4A-C).

TABLE 2

| Enzyme prepared TbPO₄ | | | | Sodium Phosphate Prepared TbPO₄ | | | |
|---|---|---|---|---|---|---|---|
| WT % | | AT % | | WT % | | AT % | |
| Element | AVG | Element | AVG | Element | AVG | Element | AVG |
| C | 4.2 | C | 16.3 | C | 2.6 | C | 8.8 |
| N | 0 | N | 0 | N | 0 | N | 0 |
| O | 16.5 | O | 48.4 | O | 22.8 | O | 57.8 |
| Na | 0.3 | Na | 0.7 | Na | 0.3 | Na | 0.5 |
| Si | 0.3 | Si | 0.5 | Si | 0 | Si | 0 |
| P | 8.3 | P | 12.6 | P | 11.2 | P | 14.7 |
| S | 0.3 | S | 0.5 | S | 1.4 | S | 1.8 |
| Tb | 70.1 | Tb | 21.0 | Tb | 60.7 | Tb | 15.5 |

Thermal gravimetric analysis (TGA) of freshly dried material was performed to determine the initial form of material was hydrated. TGA indicated that the sample was not completely dry at the start, but later, mass changes indicated triphosphate or tetraphosphate form prepared at room temperature before calcination (FIGS. 5A, B).

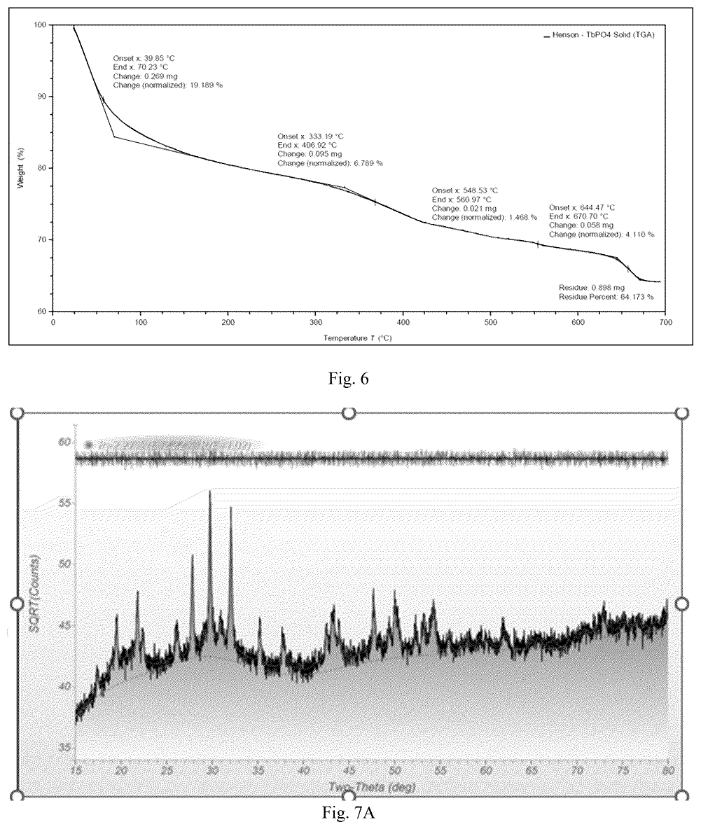

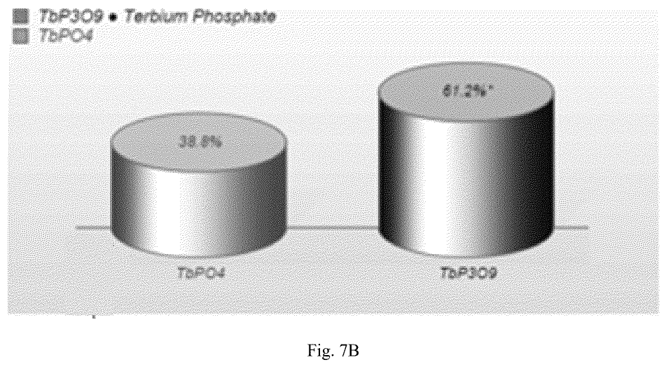

Solid, dried precipitate (30 mg) was analyzed for fluorescence with excitation at 350 nm to determine functionality without calcination. Fluorescence peaks of 492, 548, 586, and 620 nm were observed, matching results from TbPO4 nanorod synthesis (Tran et al 2012), suggesting functional crystal formation without heating is possible (FIG. 6). XRD analysis is shown in FIGS. 7A-B.

### Terbium Phosphate Protocol

1. Prepare a solution of 200 mM sodium acetate and adjust the pH to 5.0 (NaOAc MW: 82.03g/mol).
2. Divide the sodium acetate buffer solution into two containers and aliquot 1 mL into an Eppendorf tube or other appropriate container.
3. In one buffer solution bottle, dissolve an amount of terbium sulfate such that the final combined volume concentration of free terbium ions is 1 mM (500 μM of Tb₂(SO₄)₃). (Anhydrous Tb₂(SO₄)₃ MW: 750.16 g/mol).
4. In the other buffer solution bottle, dissolve an amount of glycerophosphate such that the final combined volume concentration is 1 mM (Glycerophosphate MW: 172.074 g/mol)
5. In the aliquot of buffer, dissolve an amount of acid phosphatase (phosphatase, acid from potato (cat. #5245291KU) available from Millipore Sigma) such that the final combined volume concentration is 0.05 g/L.
6. Combine the three solutions and allow to incubate a shaker at 140 rpm, 37° C. for 24 hours.
7. Filter the solution using standard methods, such as a Buchner funnel with weighed filter paper.
8. Dry on the filter paper to determine yield.

### Example 3

For the microorganisms that tolerate growth at low pH conditions, acid phosphatase activity is recombinantly expressed in the cells and the enzymatic activity is directed for secretion. For the microorganisms that grow in high pH conditions, alkaline phosphatase activities are explored. Endogenous phosphatases as annotated in the sequenced genomes of the host organisms is expressed. pH effects on the phosphorylation to achieve complete conversion of at least one REEs to insoluble phosphate complexes by tweaking pH conditions is explored. Although stability constants describing the formation of REE phosphate complexes are similar in magnitude for some REE carbonate complexes, equilibrium calculations indicate that phosphate ions likely affect dissolved REEs via precipitation of REE phosphate salts.

Release of inorganic phosphates during microbial metabolism results in the precipitation of REEs in the form of phosphate (Equation 1 and 2).

REE³⁺+HPO₄²⁻+nH₂O->REE(PO₄)×nH₂O+H⁺ (1)

REE³⁺+H₃PO₄+nH₂O->REE(PO₄)×nH₂O+3H⁺ (2)

This approach is tested with several engineered strains to compare the performance to known microorganisms e.g., Citrobacter sp. and B. megaterium that have already shown phosphate-based precipitation of Dy, Nd and Eu (50, 90 and 85%) in pH 5. Some metals may require careful tailoring of acidity, as the recovery of metals such as La is reduced due to the inefficient desolubilization of LaPO₄ in acidic conditions. This mechanism enables the development of a separation process for stepwise recovery of REEs and could be complementary to the protein binding tags.

1. A process for forming a rare earth element (REE) phosphate, the process comprising:
(a) contacting an acid phosphatase with a phosphate source in a solution, thereby generating free phosphate;
(b) contacting an REE sulfate or an REE chloride with the free phosphate formed in step (a), thereby forming an REE phosphate; and
(c) isolating the REE phosphate formed in step (b).

2. The process of claim 1, wherein the REE is Scandium (Sc), Yttrium (Y), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), or mixtures thereof.

3. The process of claim 1, wherein the REE is Europium (Eu), Lanthanum (La), or Terbium (Tb).

4. The process of claim 1, wherein the pH of the solution in step (a) is about 1 to about 7.

5. The process of claim 4, wherein the solution comprises a sodium acetate, citrate, malonate, glycine, Tris, MES (2-(N-morpholino)ethanesulfonic acid), Homopiperazine-1,4-bis(2-ethanesulfonic acid, (Homo-PIPES), sodium chloride, potassium chloride, sulfuric acid, hydrochloric acid, glutamic acid, histidine, or malate.

6. The process of claim 5, wherein the solution comprises a EDTA or EGTA.

7. The process of claim 1, wherein the phosphate source is pyrophosphate, adenosine triphosphate, or a hydroxy-substituted C₁-C₆ alkyl.

8. The process of claim 1, wherein the phosphate source provides a single phosphate per molecule.

9. The process of claim 1, wherein the phosphate source is present at a concentration of about 1 μM to about 1 M when the process begins.

10. The process of claim 1, wherein the acid phosphatase is present at a concentration of 0.02 g/L or lower.

11. The process of claim 1, wherein the process is performed in the presence of a cell.

12. The process of claim 11, wherein the cell generates the acid phosphatase.

13. The process of claim 1, wherein the process is performed at a temperature of about 4° C. to about 50° C.

14. The process of claim 13, wherein the REE phosphate is formed at a rate of about 0.001 g/l/hr to about 100 g/L/hr, preferably about 0.1-g/L/hr to about 1 g/L/hr.

15. The process of claim 1, wherein the step of isolating the REE phosphate yields crystalline REE phosphate.

16. A coating comprising REE phosphate produced by a process according claim 1.

17. A process for converting Rare Earth Elements (REEs) to phosphate forms, the method comprising:
adding a phosphatase to a sample comprising an REE salt, and
recovering an REE phosphate from the sample.

18. The process of any claim 17, wherein the phosphatase comprises a conserved domain with an active site consisting of two metal ions coordinated with octahedral geometry by a cage of histidine, aspartate, and asparagine residues.

19. The process of claim 17, wherein the phosphatase comprises SEQ ID NO.: 1.

20. The process of claim 17, wherein the phosphatase has at least about 95% sequence similarity to SEQ ID NO.: 1.


SEQUENCE LISTING (1 sequence)

SEQ ID NO: 1 - protein, 477 aa, Solanum tuberosum
MLLHIFFLLSLFLTFIDNGSAGITSAFIRTQFPSVDIPLENEVLSVPNGYNAPQQVHITQGDYDGEAVIISWVTADEPGSSEVRYGLSEGKYDVTVEGTLNNYTFYKYESGYIHQCLVTGLQYDTKYYYEIGKGDSARKFWFETPPKVDPDASYKFGIIGDLGQTYNSLSTLQHYMASGAKSVLFVGDLSYADRYQYNDVGVRWDTFGRLVEQSTAYQPWIWSAGNHEIEYFPSMGEEVPFRSFLSRYPTPYRASKSSNPLWYAIRRASAHIIVLSSYSPFVKYTPQWHWLKQEFKKVNREKTPWLIVLMHVPIYNSNEAHFMEGESMRSAYERWFVKYKVDVIFAGHVHAYERSYRISNIHYNVSGGDAYPVPDKAAPIYITVGDGGNSEGLASRFRDPQPEYSAFREASYGHSTLDIKNRTHAIYHWNRNDDGNNITTDSFTLHNQYWGSGLRRRKLNKNHLNSVISERPFSARL